(19) United States
(12) Patent Application Publication
(10) Pub. No.: US 2007/0254282 A1
(43) Pub. Date: Nov. 1, 2007

(54) Catalytic Polynucleotide and Its Use for Determination of Analytes

(75) Inventors: Itamar Willner, Mevasseret Zion (IL); Yi Xiao, Jerusalem (IL); Valeri Pavlov, Jerusalem (IL); Tamara Niazov, Holon (IL)

Correspondence Address: BROWDY AND NEIMARK, P.L.L.C.;624 NINTH STREET, NW, SUITE 300, WASHINGTON, DC, 20001-5303, US

(73) Assignee: YISSUM RESEARCH AND DEVELOPMENT COMPANY OF THE HEBREW UNIVERSITY OF JERUSALEM, Givat Ram (IL)

(21) Appl. No.: 10/588,153

(22) PCT Filed: Jan. 27, 2005
(86) PCT No.: PCT/IL05/00097

§ 371 Date: Mar. 2, 2007

Related U.S. Application Data
(60) Provisional application No. 60/539,579, filed on Jan. 29, 2004.
(60) Provisional application No. 60/539,566, filed on Jan. 29, 2004.

(51) Int. Cl. C12Q 1/68

(52) U.S. Cl. 435/6

(57) ABSTRACT

A method for the determination of an analyte in a sample, the method comprising: (a) providing a catalytic polynucleotide; (b) contacting the catalytic polynucleotide with the sample so that the catalytic polynucleotide may bind to the analyte; (c) providing assay conditions such that the catalytic polynucleotide produces an optically detectable signal in the presence of the analyte; and (d) detecting the signal, thereby determining the presence of the analyte in the sample. One application of the method is for the detection of telomerase in a biological sample.

The present application claims priority from U.S. provisional application Nos. 60/539,566 and 60/539,579, whose entire contents are incorporated by reference.

### FIELD OF THE INVENTION

This invention relates to a method for detecting an analyte in an assayed sample. More specifically, the present invention concerns catalytic polynucleotides and their use in the determination of an analyte in a liquid medium.

### BACKGROUND OF THE INVENTION

The following documents are regarded as relevant to the background of the invention:

### REFERENCES

(1) Welz, R.; Schmidt, C.; Muller, S. Mol. Biol. 2000, 34, 934-939.
(2) Muotri, A. R.; Pereira, L. D.; Vasques, L. D.; Menck, C. F. M. Gene 1999, 237, 303-310.
(3) Breaker, R. R. Nat. Biotechnol. 1997, 15, 427-431.
(4) Emilsson, G. M.; Breaker, R. R. Cell. Mol. Life. Sci. 2002, 59, 596-607.
(5) (a) Travascio, P.; Bennet, A. J.; Wang, D. Y.; Sen, D. Chem. Biol., 1999, 6, 779-787. (b) Witting, P. K. Travascio, P.; Sen, D.; Mauk, A. G. Inorg. Chem. 2001, 40, 5017-5023. (c) Chinnapen, D. J. F.; Sen, D. Biochemistry 2002, 41, 5202-5212.
(6) (a) Tyagi, S.; Kramer, F. R. Nat. Biotechnol. 1996, 14, 303-308. (b) Tyagi, S.; Marras, S. A. E.; Kramer, F. R. Nat. Biotechnol. 1998, 18, 1191-1196.
(7) Dubertret, B.; Calame, M.; Libchaber, A. Nat. Biotechnol. 2001, 19, 365-370.
(8) Fan, C. H.; Plaxcxo, K. W.; Heeger, A. J. Proc. Natl. Acad. Sci. USA 2003, 100, 9134-9137.
(9) Blackburn, E. H. Cell 2001, 106, 661-673.
(10) (a) F. Patolsky, Y. Weizmann, I. Willner, J. Am. Chem. Soc., 2002, 124, 770-772; (b) F. Patolsky, A. Lichtenstein, I. Willner, Chem. Eur. J., 2003, 9, 1137-1145.
(11) (a) F. Patolsky, E. Katz, I. Willner, Angew. Chem. Int. Ed., 2002, 41, 3398-3402; (b) Y. Weizmann, F. Patolsky, E. Katz, I. Willner, J. Am. Chem. Soc., 2003, 125, 3452-3454; (c) F. Patolsky, Y. Weizmann, E. Katz, I. Willner, Angew. Chem. Int. Ed., 2003, 42, 2372-2376.
(12) F. Patolsky, E. Katz, I. Willner, Angew. Chem. Int. Ed., 2002, 41, 3398-3402.
(13) (a) Harley, C. B.; Futcher, A. B.; Greider, C. W. Nature 1990, 345, 458-460. (b) Hastie, N. D.; Dempster, M.; Dunlop, M. G.; Thompson, A. M.; Green, D. K.; Allshire, R. C. Nature 1990, 346, 866-867.
(14) (a) Shay, J. W.; Wright, W. E. Curr. Opin. Oncol. 1996, 8, 66-71. (b) Shay, J. W.; Bacchetti, S. Eur. J. Cancer 1997, 33, 787-791.
(15) Kim, N. W.; Piatyszek, M. A.; Prowse, K. R.; Harley, C. B.; West, M. D.; Ho, P. C. H.; Coviello, G. M.; Wright, W. E.; Weinricht, S. L.; Shay, J. W. Science 1994, 266, 2011-2015.
(16) Schmidt, P. M.; Lehmann, C.; Matthes, E.; Bier, F. F. Biosens. Bioelectron. 2002, 17, 1081-1087.
(17) Patolsky, F.; Gill, R.; Weizmann, Y.; Mokari, T.; Banin, U.; Willner, I. J. Am. Chem. Soc. 2003 125, 13918-13919.

In the following, reference to the above references will be made by referral to their numbering.

The discovery of catalytic RNAs (ribozymes) sparked scientific interest directed to the preparation of new biocatalysts.^{1,2} Analogous deoxyribozymes (catalytic DNAzymes) were not found in nature, but synthetic efforts demonstrated the successful preparation of numerous catalytic DNAs for a variety of chemical transformations.^{3,4}

An interesting example of a catalytic DNA that revealed peroxidase-like activities is a supramolecular complex between hemin and a single-stranded guanine-rich nucleic acid (aptamer).⁵ This complex was reported to catalyze the oxidation of 2,2′-azinobis(3-ethylbenzothiozoline)-6-sulfonic acid, ABTS, by H₂O₂ (a common reaction used for the assay of peroxidase activity). It was suggested that the supramolecular docking of the guanine-quadruplex layers facilitates the intercalation of hemin into the complex, and the formation of the biocatalytically active hemin center.

Enzymes⁽¹⁰⁾ and, specifically, horseradish peroxidase (HRP)^{(11),(12)} are used as biocatalytic labels for the amplified detection of DNA sensing events. The electrochemical amplified detection of DNA was accomplished in the presence of different enzymes,⁽¹⁰⁾⁽¹¹⁾ and the chemiluminescence analysis of DNA was reported in the presence of HRP.⁽¹²⁾

Nucleic acids in beacon configurations are extensively used as specific DNA sensing matrices. The specific linkage of photoactive chromophores/quenchers to the hairpin termini results in the chromophore luminescence quenching. The subsequent lighting-up of the chromophore luminescence by the hybridization of the analyzed DNA hairpins and the beacons opening, was used as a general motif for the photonic detection of DNA.⁶ The quenching of dyes by molecular or nanoparticle quenchers⁷ or the fluorescence resonance energy transfer (FRET) between dyes was used for the optical detection of the hybridization process of the DNA to the beacon. Recently, the labeling of the beacon termini with redox-active units led to the electrochemical detection of hybridization to the hairpins and their ring opening.⁸ In that system, ferrocene units were linked to the end of the beacon assembled on an Au electrode. While in the hairpin structure effective electron-transfer between the ferrocene units and the electrode exists, their opening by hybridization with DNA blocks the electrical communication.

The chromosomes are protected by nucleic acids of constant repeats termed telomeres.¹³ The gradual erosion of the telomere units during cell proliferation provides a cellular signal for terminating the cell cycle. In certain cells there is accumulation of the ribonucleoprotein telomerase that incorporates the telomere units into the chromosome ends, and this turns the cells into immortal entities. Indeed, in over 95% of the different cancer or malignant cells, elevated amounts of telomerase were detected, and the monitoring of telomerase activity in cells is promising for cancer diagnostics.¹⁴ Several analytical procedures for the determination of telomerase activity were developed and these include the TRAP method¹⁵ (telomeric repeat amplification protocol), the fluorescence detection of telomerase activity,¹⁶ or the recently reported¹⁷ optical detection of telomerase using CdSe/ZnS quantum dots.

Glossary

In the context of the present invention, the term “catalytic polynucleotide” refers to any nucleic acid-based molecule or complex of molecules, which have catalytic activity. The catalytic polynucleotides may be, for example, ribozymes or DNAzymes, and in a preferred embodiment may be a G-rich nucleic acid sequence that binds hemin and thus has a peroxidase activity.

The catalytic polynucleotide may be a priori active (for example already a complex of DNAzyme and hemin), or may be activated after addition of an appropriate co-factor (hemin for DNAzyme, Mg⁺⁺ for ribozyme).

The catalytic polynucleotide may be attached (by covalent binding or any other binding) to one member of a “complex forming group” (see below) such as a sequence that is complementary to an analyte such as one or more of the repeats formed by telomerase. It should be noted that once a repeat is formed, several catalytic polynucleotides may hybridize therewith, thus significantly increasing the amount of the detectable label the sensitivity of the detection method.

The non-bound catalytic nucleotides may be removed, for example, by washing. The bound catalytic nucleotides are then detected by their catalytic activity, which may be any catalytic activity known to be catalyzed by catalytic nucleotides to produce a detectable catalytic product. For example, the activity may be cleavage, splicing, rearrangement, or phosphorylation (known in ribozymes). More specifically the activity may be peroxidase activity as known using a DNAzyme (with hemin).

The term “determination” in the context of the present invention means qualitative and/or quantitative detection.

The term “binding conditions” in the context of the present invention means any conditions that allow two or more members of a complex forming group to bind together. Such conditions may comprise concentrations and/or type of solutes, pH, temperature, presence of additional members of said complex forming group, etc. The term binding conditions may also mean several different conditions applied one after the other allowing a number of complexes to form and/or dissipate, as necessary.

The term “assay conditions” in the context of the present invention means any conditions (or sequences of conditions) that allow desired binding and catalytic activity (or a sequence of binding or catalytic activities) to take place and the desired signal to be formed. The assay conditions include providing a substrate. The substrate is any substrate of the catalyzed reaction that under assay conditions yields a reaction signal. The assay conditions may also comprise concentration or type of solutes, pH, and temperature. It is noted that in some cases care should be taken that the assay conditions should be such that would not disrupt any binding that was achieved earlier under binding conditions.

In the case of a catalytic polynucleotide having peroxidase activity, for example, the assay conditions should allow catalysis of a peroxidase reaction yielding light, color or a sediment, as the case may be. In such a case, H₂O₂ and, if necessary, a hemin moiety should be provided. It is noted that H₂O₂ may be produced directly or by providing the conditions that allow its formation in the assay solution (e.g. by doxorubicin). The substrate may be for example 2,2′-azinobis(3-ethylbenzothiozoline)-6-sulfonic acid (ABTS) or luminol. In case of light detection for a peroxidase reaction, the substrate may be any substrate of the nucleic acid peroxidase that would yield light as a result of the peroxidase reaction (e.g. luminol).

The term “complex forming group” in the context of the present invention means any group comprising two or more members having a specific affinity towards one another and thus capable of binding together (directly or indirectly) in a selective manner to form a complex. Such groups may comprise for example any of the following: an antigen and an antibody (or an antigen binding portion of an antibody), two complementary nucleic acids, a nucleic acid sequence and its binding protein, an enzyme and its substrate or inhibitor or co-enzyme, components of any known complex (e.g. components of a complex enzyme or protein complexes such as biotin-avidin), a ligand and a receptor or a ligand binding portion of a receptor, a receptor and its inhibitor, a glycoprotein and a lectin, etc. The complex forming group may comprise a plurality of binding components of different types such as a nucleic acid, its complementary nucleic acid, a protein capable of binding the formed double strand, an antibody to said protein, etc. In the context of the present invention, it may be said that the antibody and the first nucleotide sequence are part of a complex forming group and that they bind together indirectly.

The term “binding” in the context of the present invention means any type of binding in particular selective binding between molecules, including without limitation, ionic interaction, hydrogen bonds, Van der Waals interaction, covalent binding, etc.

### SUMMARY OF THE INVENTION

Thus, the present invention provides a method for determination of an analyte in a sample, the method comprising:
(a) providing a catalytic polynucleotide;
(b) contacting the catalytic polynucleotide with the sample so that the catalytic polynucleotide may bind to the analyte;
(c) providing assay conditions such that the catalytic polynucleotide produces an optically detectable signal in the presence of the analyte; and
(d) detecting the signal, thereby determining the presence of the analyte in the sample.

In a preferred embodiment, the method is characterized in that the catalytic polynucleotide is a DNAzyme and that the assay conditions comprise (also) the provision of luminol which produces in the presence of the catalytic polynucleotide a light emitting reaction.

By one aspect of the invention termed the “surface bound aspect” the analyte is immobilized on a surface, the catalytic polynucleotides is bound thereto and unbound catalytic polynucleotides are removed so that detection of the catalytic activity is indicative of the presence of the analyte.

By another aspect termed the “soluble aspect” (at times also referred to as the beacon aspect), the analyte is soluble, and its binding reverts the polynucleotide from a pre-catalytic (inactive) configuration to a catalytic (active) configuration. Said reversion is typically achieved by removal from the pre-catalytic form, through the binding of the analyte, of a steric hindrance caused by an inhibitory moiety.

The development of catalytic beacons provides a major advance in DNA sensing. The invention includes in one embodiment of the soluble aspect the tailoring of catalytic polynucleotides (beacons) for the sensing of the presence of telomerase repeats produced due to telomerase activity originating from cancer cells. Beacon structures may be designed that, upon analyte-induced removal of steric hindrance caused by an inhibitory moiety (such as upon opening the hairpin structure by hybridization with the analyte), yield in the presence of hemin a DNAzyme that allows the biocatalytic analysis of the hybridization process.

According to the surface bound aspect of the present invention, a method is provided for the detection in an assay sample of an analyte, the analyte being one member of a complex forming group, the method comprising:
(a) immobilizing the analyte on a solid surface;
(b) providing a catalytic polynucleotide bound to another member of the complex forming group;
(c) contacting the catalytic polynucleotide with the solid surface under conditions allowing binding between the two members of the complex forming group;
(d) removing unbound catalytic polynucleotide;
(e) providing assay conditions to allow the catalytic polynucleotide to catalyze a reaction yielding an optically detectable signal; and

detecting the optically detectable signal, thereby detecting the presence of the analyte in the sample.

The method of the invention, as it is based on a catalytic reaction which amplifies the original signal, is very useful for detection of analytes, especially biological molecules in a sample. The integration of a DNA biocatalyst into DNA detection schemes provides several important advantages: (i) The catalytic DNA may substitute the protein-based biocatalysts, and thus eliminate non-specific binding phenomena; (ii) Tailoring of the DNA biocatalyst as a part of the labeled nucleic acid may reduce the number of analytical steps for detection of the DNA.

The DNAzyme includes two separated nucleic acid portions that constitute the single stranded peroxidase deoxyribozyme that forms a layered G-quadruplex structure. The nucleic acids self-assemble in the presence of hemin to form a biocatalyst for the generation of chemiluminescence in the presence of H₂O₂ and luminol.

The determination according to the present invention can be qualitative detection or quantitative measurement. In both cases care must be taken that any background luminescence be disregarded. It is therefore required that the method contain a step wherein un-bound hemin and/or catalytic polynucleotide be removed from the system.

In quantitative detection, the method should also comprise a calibration step in which calibration assay samples containing known concentrations of the analyte are used in order to correlate the light emission and analyte concentration. Such calibration stages are well known in the art.

Thus, in yet another aspect of the invention, a method is provided for the quantification in an assay sample of an analyte, comprising one member of a complex forming group, using a catalytic polynucleotide, the method further comprising the following step:
(e) comparing the optically detectable signal detected in step
(d) with a calibration scale, thereby quantifying the amount of analyte in the sample.

The calibration scale may be prepared on the basis of at least two calibration standards containing known, but different, concentrations of the analyte immobilized onto a solid surface. For the purpose of producing the calibration curve, the following steps may be applied:

contacting the catalytic polynucleotide with each of the calibration standards under binding conditions;

removing from the calibration assay sample any nucleic acid peroxidase that is not bound to the analyte;

providing assay conditions such that in the presence of the analyte light is emitted;

detecting light from each calibration assay sample; and

deducing a calibration scale.

The determination of the analyte in the assay sample may be carried out by:

providing an assay sample;

contacting the catalytic polynucleotide with the assay sample under binding conditions;

removing from the assay sample any catalytic polynucleotide that is not bound to the analyte;

providing assay conditions such that in the presence of the analyte light is emitted;

detecting the light, thereby detecting the presence of the analyte in the assay sample; and

comparing the light detected in the assay sample with the calibration scale, thereby quantifying the amount of analyte in the assay sample.

In accordance with the surface bound aspect of the present invention, the complex comprising the analyte and the nucleic acid peroxidase may be bound to a solid surface such as a plate, slide, chip, bead or bead-like structure, magnet, etc. This may, for example, facilitate removal of the unbound catalytic polynucleotide, as is known in the art. The presence or amount of bound catalytic polynucleotide thus correlates to the presence or amount, respectively of the analyte in the sample.

The analyte may be immobilized to the solid surface by specific or non specific interactions such as by the use of immobilized antibodies or immobilized sequences complementary to part of the analyte, immobilized poly T sequences complimentary to poly A of RNA sequences and in general by relatively non-specific manners known to immobilize the type of molecule of the analyte to a solid surface.

The determination according to the present invention as described above and as exemplified below may be sensitive enough to differentiate between two nucleotide analytes having only one base pair difference between them.

In the present invention as described in the methods detailed above, the light emitted in accordance with the presence invention may be enhanced or amplified by providing a plurality of catalytic polynucleotides (at least one of which comprises a member of the complex forming group) being bound to a bead like particle (e.g. gold). Thus, when the catalytic polynucleotides, being a member of the complex forming group, is bound to an analyte, the other catalytic polynucleotides are also bound thereto via the bead like particle and are not removed from the sample along with un-bound catalytic polynucleotide. Thus all the catalytic polynucleotides present on the bead-like particle may contribute to the production of the light emitting signal. Accordingly the detection methods of the present invention have two steps of signal amplification: one in that each catalytic polynucleotide can catalyze a plurality of light emitting products and one that several such catalytic polynucleotides become bound for each analyte molecule.

In accordance with the soluble (beacon) aspect of the present invention, a method is provided for detection of an analyte being one member of a complex forming group in an assay sample, the method comprising:
(a) providing a pre-catalytic polynucleotide comprising a catalytic polynucleotide moiety attached to an inhibitory moiety comprising another member of the complex forming group, the inhibitory moiety in the absence of the analyte sterically hindering the catalytic activity of the catalytic polynucleotide while in the pre-catalytic complex, and the steric hindrance being removed upon binding of the inhibitory moiety to the analyte;
(b) contacting the pre catalytic complex with the assay sample under binding conditions;
(c) providing assay conditions which allow the catalytic polynucleotide to catalyze a reaction yielding an optically detectable signal; and

detecting the signal, thereby detecting the presence of the analyte in the assay sample.

In a preferred embodiment of this aspect of the invention, the analyte is a nucleic acid analyte, and the complex forming groups comprise nucleotide sequences being complementary one to the other.

In this aspect the inhibitory moiety is a nucleic acid sequence that in the absence of the analyte sterically hinders (for example due to intra-hybridization) the catalytic polynucleotide. Only where the analyte is present the inhibitory sequence hybridizes therewith sterically changing its position and removing the inhibition thus enabling the catalytic polynucleotide to exert its catalytic activity.

In a further aspect of the present invention, a method is provided for detection in an assay sample of an analyte being one member of a complex forming group, the method comprising:

providing two or more nucleic acid sequences forming a catalytic nucleic acid complex having, in the absence of an analyte, catalytic activity which produces a detectable product, at least one of the two or more nucleic acid sequences attached to another member of the complex forming group, such that binding of the analyte to the other member prevents the catalytic activity,

contacting the two or more nucleic acids with the assay sample under binding conditions;

providing assay conditions such that in the absence of the analyte the detectable product is produced; and

detecting reaction signal and comparing the reaction signal to a control signal produced in the absence of the analyte, a decrease of the reaction signal indicating the presence of the analyte in the assay sample.

The determination according to this aspect of the present invention can be qualitative detection or quantitative measurement. In quantitative detection, the method should also comprise a calibration step in which calibration assay samples containing known concentrations of the analyte are used in order to correlate the reduction in reaction signal emission and analyte concentration. Such calibration stages are well known in the art.

Thus, in yet another aspect of the invention, a method is provided for the quantification of an analyte being one member of a complex forming group in an assay sample, using two or more nucleic acids forming a catalytic nucleic acid complex, at least one of the two or more nucleic acids comprising another member of the complex forming group, such that binding of the analyte to the at least one of the two or more nucleic acids prevents the catalytic activity, the method comprising:

providing the two or more nucleic acids;

preparing a calibration scale, the preparation comprising:

providing at least two calibration assay samples containing known and varying concentrations of the analyte;

contacting the two or more nucleic acids with each of the calibration assay samples under binding conditions;

providing assay conditions such that in the absence of the analyte a reaction signal is emitted;

detecting the reaction signal from each calibration assay sample;

deducing a calibration scale;

detecting presence of the analyte in the assay sample, the detection comprising:

providing an assay sample;

contacting the two or more nucleic acids with the assay sample under binding conditions;

providing assay conditions such that in the absence of the analyte a reaction signal is emitted;

detecting the reaction signal from the assay sample;

comparing the reaction signal detected in the assay sample with the calibration scale, thereby quantifying the amount of analyte in the assay sample.

The “reaction signal” may be any measurable parameter (or product yielded as a result of the catalyzed reaction. The term “reaction” is used to denote one or more reactions or interactions carried out at once or in sequence, to yield the reaction signal. The reaction signal may thus be an electric response including any measurable change in the electrical parameters recorded by or electrical properties of the electrode. An electric response may be flow of current, charge or potential change that results from the reaction occurring at the surface of the electrode; a change in the amperometric response of the electrode that can be measured, for example, by means of a cyclical voltamogram; etc. In addition to an electric response, other examples for the reaction signal are the emission of light, a colorimetric response or the formation of a precipitate on a sensing member.

The invention is not limited by the manner in which the reaction signal is measured and any manner of measurement that may be used therefore could be applied for measurement of the electric response in the method of the invention.

In one aspect, each of the two or more nucleic acids forming a catalytic nucleic acid complex also comprises a member of a complex forming group whilst the analyte comprises the other member. In such case, under binding conditions, the analyte may bind (simultaneously or separately) the two or more nucleic acids.

According to a preferred embodiment of the above method, the catalytic nucleic acid complex is a polynucleotide peroxidase, formed of at least two nucleic acid fragments of a single strand nucleic acid molecule forming the polynucleotide peroxidase. The two or more nucleic acids may form together the whole sequence of the polynucleotide peroxidase (e.g. obtainable by splitting the polynucleotide peroxidase in two) or at least an active part thereof (e.g. if some bases are removed).

Thus according to a preferred embodiment the present invention provides a method for detection of a nucleic acid analyte being one member of a complex forming group in an assay sample, the method comprising:

providing the two or more nucleic acid sequences forming together a nucleic acid peroxidase, at least one of the two or more nucleic acids comprising another member of the complex forming group, such that binding of the analyte to the at least one of the two or more nucleic acid sequences prevents the peroxidase activity;

contacting the two or more nucleic acids with the assay sample under binding conditions;

providing assay conditions such that in the absence of the analyte a reaction signal is emitted;

detecting the reaction signal, thereby detecting the presence of the analyte in the assay sample.

The reaction signal in this case may be for example any measurable parameter resulting, directly or indirectly, form the peroxidase reaction. Examples for such signals are a calorimetric product (wherein the substrate is ABTS) or light (wherein the substrate is luminol).

In one aspect of the present invention, the nucleic acid complex may be bound to a solid surface such as a plate, slide, chip, bead or bead like structure, magnet, etc. This may, for example, allow measurement of the nucleic acid complex' activity via measurement of a precipitate resulting from the catalyzed reaction, as is known in the art.

Accordingly another aspect of the invention provides a method for detection in an assay sample of an analyte being one member of a first complex forming group, the method comprising:

providing the two or more nucleic acid sequences forming a catalytic nucleic acid complex, at least one of the two or more nucleic acids comprising another member of the first complex forming group, and at least one of the two or more nucleic acids comprising a member of a second complex forming group, such that binding of the analyte to the at least one of the two or more nucleic acids prevents the catalytic activity;

providing a solid surface having bound thereto a member of the second complex forming group and contacting the solid surface with the two or more nucleic acids under binding conditions such that the nucleotide peroxidase becomes bound to the solid surface;

contacting the solid surface with the assayed sample under binding conditions such that the analyte binds to the at least one of the two or more nucleic acids that comprises the other member of the first complex forming group;

providing assay conditions such that in the absence of the analyte a reaction signal is emitted;

detecting the reaction signal, thereby detecting the presence of the analyte in the assay sample.

Another aspect of the invention relates to the detection of telomerase in a biological sample, using catalytic polynucleotides.

The detection of the activity of the catalytic polynucleotide may be achieved by one of two manners. In accordance with one embodiment, the detection is achieved by surface detection of products produced by immobilized catalytic polynucleotides. By another embodiment, the detection is by solely soluble components (“beams”) which in the presence of the repeat sequence produced by the telomerase revert from a pre-catalytic to a catalytic form.

By the first “surface detection” aspect, the present invention concerns a method for detection of telomerase in a sample, the method comprising:

providing a primer for telomerase activity immobilized on a solid surface;

contacting the sample with the immobilized primer in the presence of deoxynucleoside triphosphoric acids (dNTP's), under conditions enabling formation of a telomere repeat unit;

adding a catalytic polynucleotide, attached to a sequence complementary to the telomere repeat unit;

removing unbound catalytic polynucleotides;

providing substrates for catalytic polynucleotides; and

detecting the presence of catalytic products of catalytic polynucleotides, the products indicating the presence of telomerase in the sample.

The term “sample” refers to any sample suspected of having active telomerase therein, such as body fluids (blood, plasma, urine, cerebrospinal fluid, saliva, semen) or medium in which cells, suspected of having telomerase activity, have been incubated or lysed.

The “primer” is any primer on which telomerase elongation activity may take place and in particular is:

5′ TTTTTTAATCCGTCGAGCAGAGTT

The primer may be immobilized on any surface such as walls of a vessel, beads, etc. by any means known for immobilization of nucleic acid sequences.

The term “telomerase repeat unit” refers to the minimal unit which the telomerase adds to the primer. This term can refer to several such units consecutively connected to each other.

By one option the peroxidase activity may be the H₂O₂-mediated oxidation of 2-2′ azinobis (3-ethylbenzothiozoline)-6-sulfonic acid (ABTS).

By a preferred embodiment the peroxidase activity is the generation of chemiluminescence in the presence of H₂O₂ and luminol, and the detection is of chemiluminescence. Detection of said chemiluminescence is indicative to the presence of immobilized catalytic polynucleotides and hence the presence of telomerase generated repeat units, indicating the presence of catalytically active telomerase in the sample.

By a second “soluble component” aspect (also termed “beacon”) the present invention concerns a method for detection of the presence of catalytically active telomerase in a sample, the method comprising:
(a) providing a pre-catalytic polynucleotide comprising a catalytic polynucleotide attached to an inhibitory moiety, said inhibitory moiety being complementary to a telomere repeat unit, the inhibitory moiety, in the absence of the telomere repeat unit, inhibiting the catalytic activity of the catalytic polynucleotide while in the pre-catalytic polynucleotide, the pre-catalytic polynucleotide further comprising a primer for telomerase elongation;
(b) contacting the pre-catalytic polynucleotide with the sample in the presence of dNTPs and under conditions enabling formation of one or more telomere repeat units;
(c) providing substrates for the catalytic polynucleotide; and
(d) detecting an optically detectable signal of the catalytic polynucleotide, detection of the signal being indicative of the presence of telomerase in the sample.

Optionally, between steps (b) and (c) there is an additional step of providing a co-factor required for the catalytic nucleic acid activity. Examples of co-factors are hemin where the catalytic nucleic acid is DNAzyme and Mg++ and where it is a ribozyme.

The inhibitory moiety is designed for example to produce a “hairpin” configuration with part of the catalytic polynucleotide, such a configuration (produced by hybridization of the catalytic polynucleotide and the inhibitory moiety) rendering the whole complex inactive.

The pre-catalytic polynucleotide also includes a primer for telomerase activity, for example attached either to the free end of the catalytic polynucleotide or attached to the free end of the inhibitory moiety.

Production of a telomere repeat unit causes the inhibitory moiety to hybridize with the newly formed repeat (instead of hybridizing with the catalytic polynucleotide) thus “opening” the pre-catalytic polynucleotide and rendering it active (optionally together with a co-factor such as hemin).

### BRIEF DESCRIPTION OF THE DRAWINGS

In order to understand the invention and to see how it may be carried out in practice, some preferred embodiments will now be described, by way of non-limiting example only, with reference to the accompanying drawings, in which:

FIG. 1 shows a scheme illustrating two embodiments of the invention. FIG. 1A illustrates analysis of DNA by opening of a beacon nucleic acid and the generation of a DNAzyme, while FIG. 1B illustrates analyzing telomerase activity by a functional DNA beacon that self-generates a DNAzyme;

FIG. 2 shows the results of experiments carried out according to the scheme illustrated in FIG. 1A. Absorbance changes are shown originating from the formation of (4) upon analysis of: (a) (2), 4.3 μM. (b) color generated by hemin and (2), 4.3 μM, in the absence of (1). (c) Color formed by hemin and (1) without (2). (d)-(f) Analysis of variable concentrations of (2) corresponding to 3.0 μM, 2.15 μM, 1.30 μM, 0.40 μM and 0.2 μM, respectively. (i) and (j) The analysis of the SPM mutations (2a) or (2b), 4.3 μM. All experiments were performed in the presence of (1), 0.43 μM, hemin, 0.43 μM, ABTS, 3.2 mM and H₂O₂, 3.2 in M in a 0.1 M tris buffer solution, pH=8.1 that included MgCl₂ 20 mM. Inset: Calibration curve corresponding to absorbance of the system upon analyzing variable concentrations of (2) after a fixed time-interval of 60 seconds;

FIG. 3 shows the results of experiments carried out according to the scheme illustrated in FIG. 1B. Absorbance changes are shown upon analyzing telomerase activity originating from: (FIG. 3A) 10,000 HeLa cells, (FIG. 3B) 10,000 heat-treated HeLa cells (95oC, 10 minutes). In all experiments the systems consisted of the catalytic beacon (5), 0.04 μM, hemin, 0.04 μM, ABTS, 3.2 mM and H2O2, 3.2 mM in 0.1 tris buffer solution, pH=8.1 that included MgCl2, 20 mM. Inset: Calibration curve corresponding to absorbance change of the system upon analyzing variable numbers of HeLa cells;

FIGS. 4, 5 and 6 show schemes illustrating further embodiments of the invention. FIG. 4A shows the suggested G-quadruplex/hemin structure of the DNAzyme. FIG. 4B shows chemiluminescence generated by a nucleic acid/hemin supramolecular complex and the inhibition of the DNAzyme activity by hybridization. FIG. 5 shows the reconstitution of nucleic acids on a hemin monolayer modified surface and the generation of a biochemiluminescence DNAzyme and the inhibition of the process by hybridization. FIG. 6 shows the assembly of a nucleic acid/hemin complex on an electrode for the electrochemical generation of chemiluminescence and the inhibition of the process by hybridization;

FIG. 7 shows the results of experiments carried out according to the scheme illustrated in FIG. 4. Integrated photons recorded in the systems consisting of: (a) The nucleic acids (1) and (2) each 12.5 μM, and hemin 12 μM. (b) Hemin 12 μM, (c) The nucleic acids (1) and (4) 12.5 μM, and hemin, 12 μM. (d) (4) 12.5 μM, without hemin. (e) to (h) (1) and (2) 12.5 μM each, hemin 12 μM and competitive hybridizing nucleic acid (4) at concentrations that correspond to 3 μM, 6 μM, 9.5 μM and 12.5 μM, respectively. (i) (4) 12.5 μM and hemin 12 μM without added (1) and (2);

FIG. 8 shows the results of experiments carried out according to the scheme illustrated in FIG. 5. Integrated photons emitted by (a) The hemin-modified surface reconstituted with (1) and (2), 2.5 μM each. (b) The hemin-modified surface without the reconstitution with (1) and (2). (c) The hemin-modified surface reconstituted with (1) or (2), 2.5 μM each. (d) to (g) The hemin-modified surface reconstituted with (1) and (2), 2.5 μM each in the presence of (4) at concentrations corresponding to 0.6 μM, 1.2 μM, 1.9 μM, 2.5 μM, respectively;

FIG. 9 shows the results of experiments carried out according to the scheme illustrated in FIG. 6. Time-dependent photons counted in the system consisting of: (a) The (6)/(7)-functionalized electrode treated with hemin, 1.2 μM, and doxorubicin, (5), 5 μM. (b) The (6) -functionalized electrode. (c) The (6)-functionalized electrode hybridized with (8), 2.5 μM. In experiments (b) to (d) the resulting electrodes were treated with hemin, 1.2 μM, and doxorubicin, 5 μM. Inset: The calibration curve that corresponds to the percent of photons as a function of the different concentrations of (8) corresponding to 0 μM, 1.2 μM, 1.9 μM, 2.5 μM, respectively. Light emission was detected upon the application of the potential corresponding to −0.6 V vs. SCE;

FIG. 10 shows the method of the invention wherein a plurality of nucleic acid peroxidases are present on a “bead-like” gold structure for amplification of the signal;

FIG. 11 shows the light emission reading with varying concentrations of the “bead-like” gold structure described in FIG. 10;

FIG. 12 is a schematic drawing illustrating the analysis of telomerase activity using DNAzyme labels and chemiluminescence as a detection signal; and

FIG. 13 shows the integrated light intensities corresponding to: (a) The analysis of 10,000 HeLa cells using the DNAzyme (7), 2.5 μM. (b) The analysis of 10,000 heat-treated HeLa cells in the presence of the DNAzyme, 2.5 μM. (c) The analysis of 10,000 HeLa cells without the DNAzyme but upon interaction with hemin 2.5 μM. (d) to (f) The analysis of 5000, 2500 and 1000 HeLa cells, respectively. Inset: Calibration curve corresponding to the analysis of variable numbers of cells.

### DETAILED DESCRIPTION OF THE INVENTION

0Materials and Methods

Hemin was purchased from Porphyrin Products (Logan, Utah), and used without further purification. A hemin stock solution was prepared in DMSO, and diluted in DMSO. Solutions were frozen and stored in the dark at −20° C. The concentration of hemin solutions was determined using standard spectroscopic methods. ^{[11]} 5-Amino-2,3-dihydro-1,4-phthalazinedione (luminol) and other chemicals were obtained from Sigma and used as supplied. All buffers used for analyzing the DNAzyme chemiluminescent activities contained the non-ionic detergent Triton X-100 (0.05%, w/v) and 1% DMSO.

Nucleic acids were synthesized by Sigma Genosys. They were purified using the PAGE method. The sequences of the oligomers are given below:

| | |
|---|---|
| (1) | 5′-CGATTCGGTACTGGCTCAAAATGRGGAGGGT-3′ |
| (2) | 5′-AGGGACGGGAAGAAAGATAATGCGCATGCTCAA-3′ |
| (4) | 5′-TGAGCATGCGCATTATCTGAGCCAGTACCGAATCG-3′ |
| (6) | 5′-HS(CH₂)₆CGATTCGGTACTGGCTCAAAATGRGGAGGGT-3′ |
| (7) | 5′-AGGGACGGGAAGATGAGCCAGTACCGAATCG-3′ |
| (8) | 5′-TGAGCCAGTACCGAATCG-3′ |

Preparation of DNA-Hemin Complexes. Nucleic acid (1), 25 μM, nucleic acid (2), 25 μM, and competitive hybridizing nucleic acid (4) at concentrations that correspond to 0 μM, 6 μM, 12 μM, 19 μM and 25 μM, respectively, were heated to 95° C. for 9 min in 10 mM Tris-HCl, pH 7.4, to dissociate any intermolecular G-quadruplex, and allowed to cool to room temperature. An equal volume of the hybridization buffer (50 mM HEPES, 40 mM KCl, 400 mM NaCl, 0.1% Triton X-100 and 2% DMSO, pH 7.4) was added to the mixtures of the nucleic acids and the systems were allowed to hybridize and fold overnight at room temperature. Hemin, 12 μM, was then added to the systems (final DMSO % less than 2%) to form the G-quadruplex structures (12 h, room temperature).

Immobilization of Hemin as a Monolayer and the Reconstitution of DNAzyme on the Gold Surface. The Au-coated (50 mm gold layer) glass plate (22 mm×11 mm) was immersed into a piranha solution (consisting of 70% concentrated sulfuric acid and 30% hydrogen peroxide) for 20 min, and afterwards thoroughly rinsed with triple-distilled water. The plate was then soaked in concentrated nitric acid for 5 min, and rinsed with water again. The plate was treated with an ethanol solution of 3-mercaptopropionic acid, 1×10⁻² M, that contained 6-mercaptohexanol, 1×10⁻³ M, for 12 h, and afterwards rinsed with ethanol to remove any non-specifically adsorbed material. The covalent coupling of 1,10-diaminodecane to the thiol monolayer-modified plate was performed by soaking the plate in 0.01 M HEPES buffer solution, pH 7.2, that included 1,10-diaminodecane, 5×10⁻⁴ M, and EDC, 1×10⁻³ M, for 2 h at room temperature. The resulting plate washed with 0.01 M HEPES buffer (pH 7.2), and incubated in 0.01 M HEPES buffer solution (pH 7.2) that included hemin, 5×10⁻⁴ M; Triton X-100, 0.05%; DMSO, 1% and EDC, 1×10⁻³ M, for 4 h at room temperature. The resulting plates were then rinsed with the hybridization buffer. A mixture of nucleic acid (1), 25 μM, and nucleic acid (2), 25 μM, was heated to 95° C. for 9 minutes in 0.01 M Tris buffer (pH 7.4), and allowed to cool to room temperature. An identical volume of the hybridization buffer was added to the nucleic acid mixture to allow proper folding (12 h, room temperature) The hemin-modified electrode was then immersed in the nucleic acid solution, 2.5 μM, for surface reconstitution (12 h, room temperature). The resulting surface reconstituted hemin/nucleotide complex layer was then interacted with (4) (0 μM, 0.6 μM, 1.2 μM, 1.9 μM and 2.5 μM) in a 0.1 M phosphate buffer that included 25 mM HEPES, 20 mM KCl, 200 mM NaCl, 0.05% Triton X-100 and 1% DMSO, pH 7.4 (12 h, room temperature).

Immobilization of (6) on the Au-surface and Its hybridization with (7) or (8). The Au-plate was reacted with a 0.4 M phosphate buffer solution, pH 7.4, of (6), 6 μM, (12 h), and the resulting surface was then treated with a 0.1 M phosphate buffer solution of 1-mercaptohexanol, 1 mM (1 h). The resulting monolayer-functionalized surface was then treated with the complementary nucleic acids (7), 2.5 μM, and (8), various concentrations that correspond to 0 μM, 1.2 μM, 1.9 μM and 2.5 μM in a solution composed of 0.1 M phosphate buffer and the perfect Hyb™ hybridization buffer (Sigma), 1:1, 5 h, to yield the ds-DNA assembly on the surface. The resulting surfaces were rinsed with the hybridization buffer and immersed in a 1.2 μM hemin buffer solution that included 25 mM HEPES, 20 mM KCl, 200 mM NaCl, 0.05% Triton X-100 and 1% DMSO, pH 7.4 (12 h, room temperature). The resulting system was further treated with doxorubicin (5), 5 μM in 0.1 M phosphate buffer, pH 7.4 (1 h, room temperature).

Light Emission Measurements. Light emission was performed by using a photon counting spectrometer (Edinburgh Instruments, FLS 920) equipped with a cooled photomultiplier detection system, connected to a computer (F900 v. 6.3 software). Before the samples analyses the background light was recorded and integrated and this was subtracted from the recorded integrated spectra of the respective samples. Sample analyses were performed by taking 15 μL of the respective DNAzyme solution or the respective modified surfaces into a cuvette that included 3.3 mL of a buffer solution consisting of 25 mM HEPES, 20 mM KCl and 200 mM NaCl, pH=9.0, that included 0.5 mM luminol and 30 mM H₂O₂.

### EXAMPLES

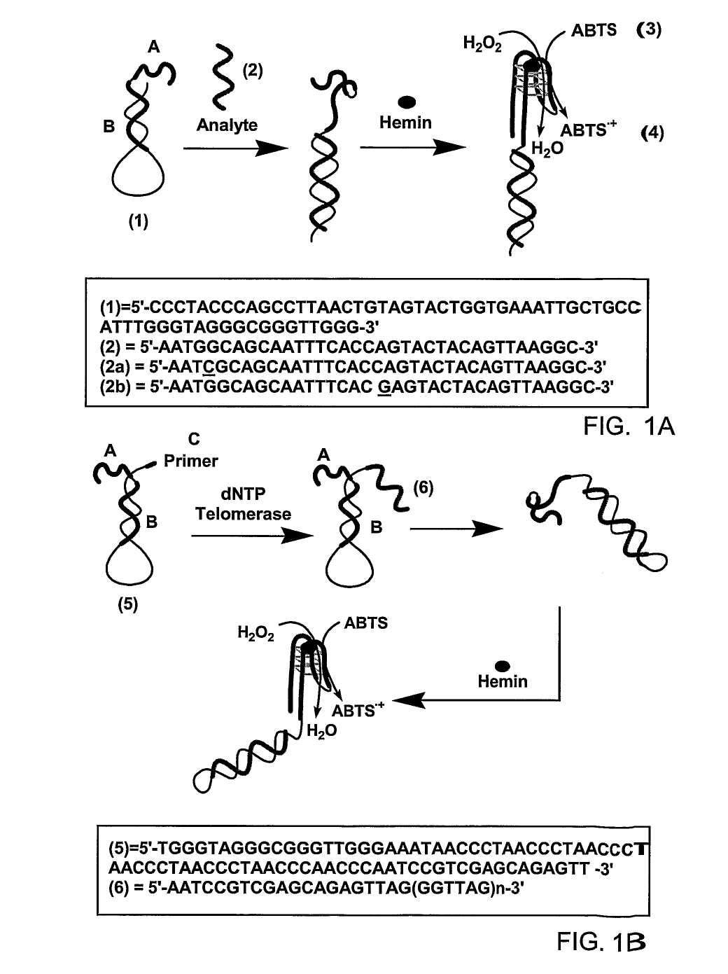

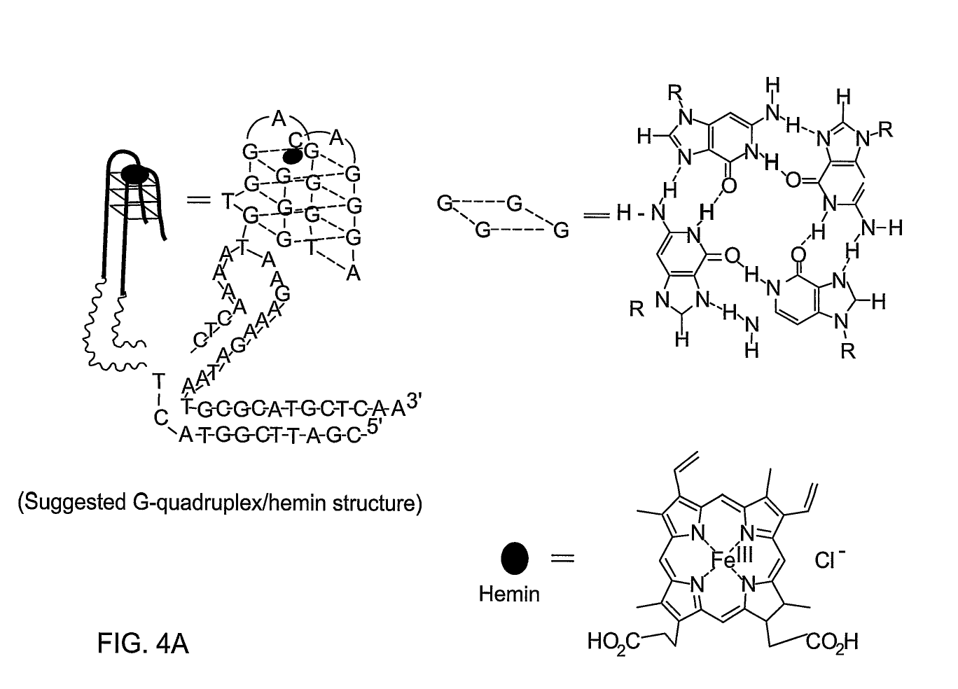

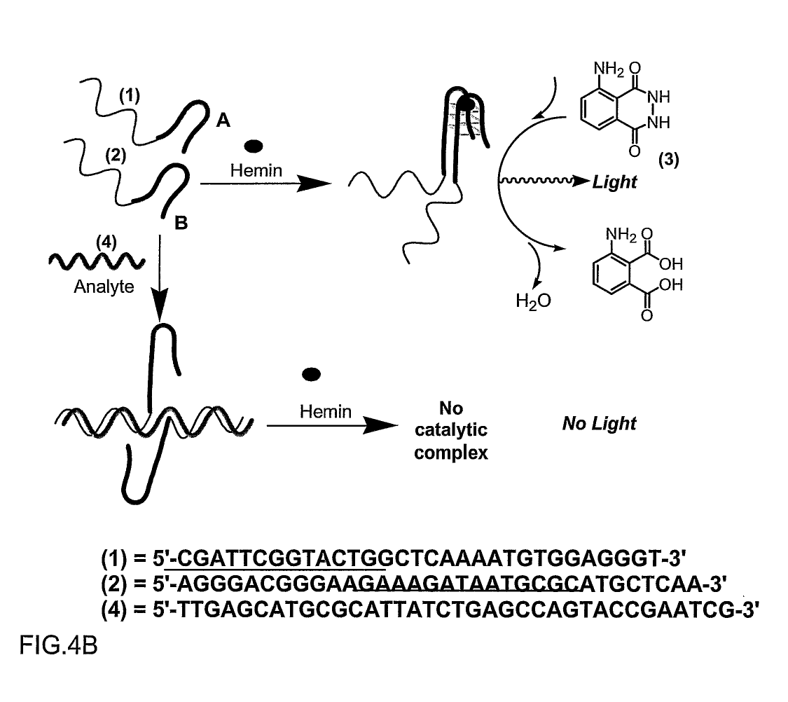

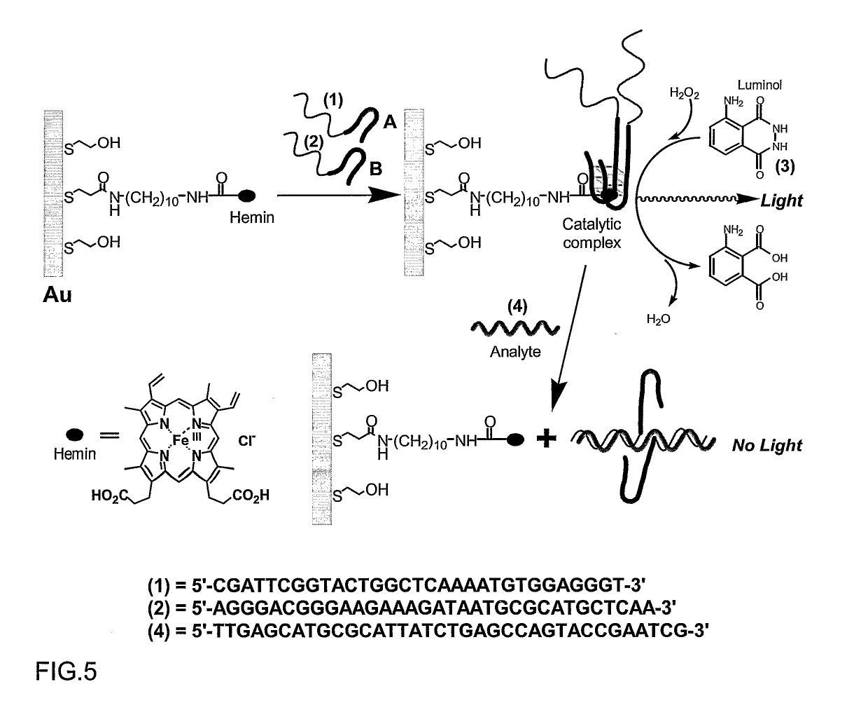

FIG. 1A depicts the method for the application of the beacon (1) as a catalytic unit for the sensing of DNA (2). The hairpin structure of (1) includes the sequence consisting of segments A and B that in an open configuration forms a G-quadruplex complex with hemin that reveals peroxidase-like activity (see FIG. 4A). Since the segment B is hybridized in the hairpin structure, the formation of the catalytic DNAzyme is prohibited. Hybridization of the analyzed DNA, (2), with the hairpin structure opens the beacon and the released sequence of nucleic acids (components A and B) self-assemble in the presence of hemin to form the catalytic DNA that catalyzes the oxidation of 2,2′-azinobis(3-ethylbenzothiozoline)-6-sulfonic acid, ABTS, (3), to the colored product (4) by H₂O₂. The hybridization and hairpin opening is then detected spectroscopically by following the accumulation of (4) at λ=414 nm (ε=36 mM⁻¹ cm⁻¹).

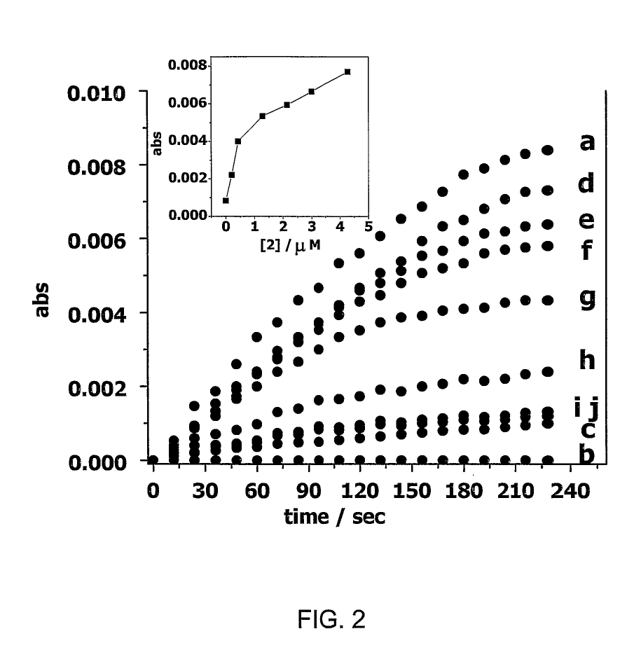

FIG. 2, curve (a), shows the time-dependent color evolution upon the analysis of the DNA (2) 4.28 μM. The control experiment that follows the spectral changes of the hairpin (1) in the presence of hemin, H₂O₂ and ABTS, does not lead to any development of a color due to the oxidation of (3). Also, the hybridization of (2) with a hairpin structure that lacks the B segment in the “hairpin stem” does not lead to an active DNAzyme. These results clearly indicate that only upon the hybridization of (2) with the beacon (1) and its opening, is the DNAzyme that stimulates the oxidation of ABTS generated. The extent of opening of the sensing beacons, and thus, the quantity of the generated DNAzyme, is controlled by the concentration of (2). FIG. 2, curves (c) to (h) show the time-dependent evolution of the oxidized product (4), at variable concentrations of the analyzed DNA. As the concentration of (2) increases, the formation of (4) is enhanced.

FIG. 2, inset, shows the extracted calibration curve that shows the color developed by the system upon analyzing variable concentrations of (2) and monitoring the color accumulated by the biocatalyzed oxidation of (3) after a fixed time-interval of 60 seconds. As expected, the biocatalytic process is enhanced as the concentration of (2) increases. The catalytic beacon reveals specificity and single base mismatches may be discerned. For example, FIG. 2, curves (i) and (j) show the time-dependent accumulation of (4) upon analyzing the mutants (2a) and (2b), 4.28 μM that include a single-base mismatch relative to the fully complementary analyzed DNA, (2). Clearly, the signal for analyzing (2) is 8-fold higher than the signal for the mutants (after 4 minutes of product accumulation).

0FIG. 1B depicts the method to analyze telomerase activity by means of a catalytic beacon. The beacon, (5), is designed to include at its two termini two functional nucleic acid components. One end of the hairpin structure ends with a nucleic acid that includes the base sequence that is a part of the DNAzyme in the presence of hemin (part A). The second part of the DNAzyme base sequence (part B) is “hidden” in the hybridized hairpin configuration. At the other end of the hairpin, a nucleic acid segment that is a primer (6) for telomerase, and for the initiation of the telomerization, is tethered to the beacon (part C of the beacon). The single stranded loop of the beacon is complementary to the telomere repeat units. Treatment of the beacons with HeLa cancer cell extract in the presence of the dNTP nucleotide mixture, results in the telomerization of the hairpin end. The elongated telomere self-generates the sequence for its hybridization with the complementary hairpin loop, and leads to the beacon opening, and to the generation of the DNAzyme. Thus, the telomerase activity is monitored by following the ABTS oxidation by H₂O₂ upon the hairpin structure opening.

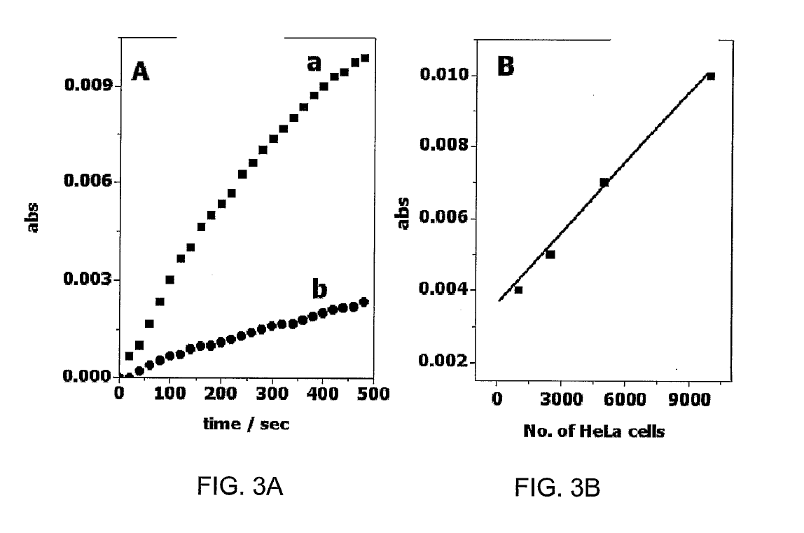

FIG. 3, curve (a), shows the time-dependent accumulation of the colored product (4) upon analyzing telomerase originating from 10,000 cells. FIG. 3, curve (b), shows the results of the control experiment where the accumulation of (4) from a system that included heat-treated (95° C. for 10 minutes) of 10,000 HeLa cell extract (the telomerase in the cells is deactivated upon heating). Clearly, the DNAzyme is not formed, and no color of (4) is developed in the system. The rate of the telomeres' formation is controlled by the content of telomerase in the sample, and thus the accumulation of (4) is regulated by the number of HeLa cells that are analyzed. 0FIG. 3, insert, shows the absorbance values of (4), obtained upon the analysis of the telomerase activity originating from different numbers of HeLa cells. (The absorbance of generated (4) is determined after a fixed time of telomerization corresponding to 8 minutes).

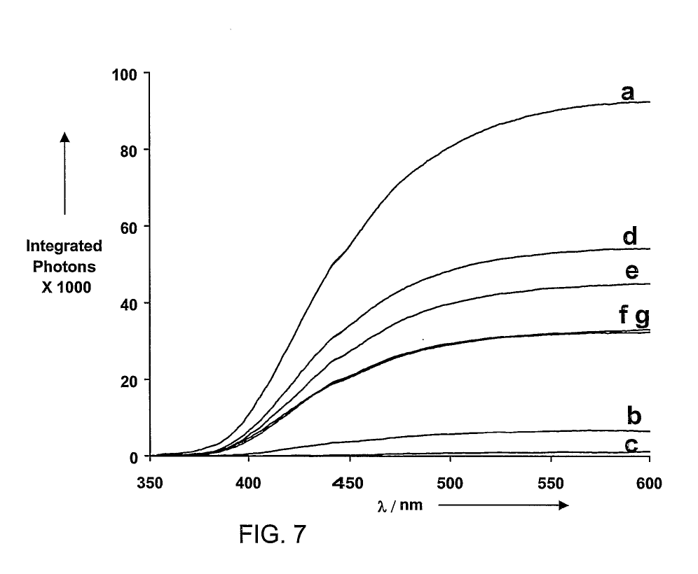

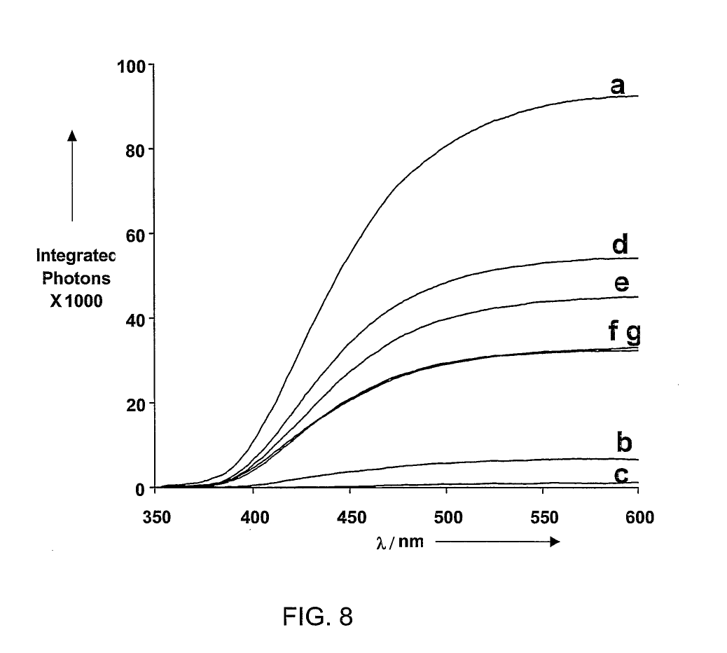

The two nucleic acids in (1) and (2) include the segments A and B that could self-assemble on the hemin site. Treatment of hemin (12 μM) with the two nucleic acids (1) and (2), 12.5 μM each, results in the formation of a 1:1:1 supramolecular complex, K_{d}=130 μM², 0FIGS. 4A and 4B. This supramolecular complex reveals biocatalytic functions and in the presence of H₂O₂ and luminol, (3), the system generates chemiluminescence. FIG. 7, curve (a), shows the integrated light intensity emitted from the system. Control experiments reveal that the formation of the supramolecular complex is essential to generate the light emission. Hemin itself yields very low chemiluminescence, curve (b), and hemin in the presence of the separated nucleic acids (1) and (4), generates a very low light output, curve (c). (The hybridization of (2) and (4) in the presence of hemin leads to negligible light emission). Also, the nucleic acid (4) in the absence of hemin does not yield any light emission, curve (d). These results indicate that the self-assembly of (1) and (2) with hemin is essential to generate the biocatalyzed light emission. The nucleic acid chains linked to the segments A and B of (1) and (2) are complementary to the 5′ and 3′ ends of the nucleic acid (4). FIG. 7, curves (e) to (h) show the effect of hybridization of (4) with the nucleic acids (1) and (2) in the presence of hemin on the emitted light intensity. Evidently, as the concentration of (4) increases, the biocatalytic light emission decreases, and at a concentration of 12.5 μM, 70% of the original chemiluminescence is blocked. A control experiment that examined the light emission from the nucleic acid (4) in the presence of hemin revealed low level chemiluminescence, 0FIG. 7, curve (i). Thus, the decrease in the chemiluminescence generated by the system consisting of the supramolecular complex hemin/(1)/(2) upon addition of (4) is attributed to the separation of the biocatalytically-active DNAzyme upon hybridization to (4). Presumably, hybridization of (1) and (2) with (4) distorts the segments A and B to a configuration that cannot form the biocatalyst structure.

The biocatalytic generation of chemiluminescence was also examined on the surface. Hemin was covalently linked to an Au-surface, 0FIG. 5. The tethered hemin units were then used as sites for the reconstitution of the biocatalytic peroxidase-like supramolecular complex on the surface, by the interaction of the functionalized surface with nucleic acids (1) and (2). Coulometric assay of the redox-wave of the heme units indicates a surface coverage of 3.5×10⁻¹¹ mole·cm⁻². Thus, ca. 18% of the hemin units are reconstituted with the nucleic acids (1) and (2). 0FIG. 8, curve (a), shows the integrated light intensity emitted by the DNAzyme interface in the presence of H₂O₂ and luminol. Control experiments confirm that very low light emission is stimulated by the hemin monolayer alone, curve (b), and that hemin in the presence of (1) or (2) alone does not lead to any significant chemiluminescence, curve (c). 0FIG. 8, curves (d) to (g), show the light emitted from the system in the presence of different concentrations of added (4). As the concentration of (4) increases, the emitted light intensity decreases. Microgravimetric quartz crystal microbalance experiments indicate that the hybridization of the free nucleic acid parts of (1) and (2) with (4) leads to the dissociation of the hemin-nucleic acids complex from the surface, and at a concentration of (4) that corresponds to 2.5 μM, the crystal frequency is almost similar to the hemin-monolayer-functionalized crystal prior to the reconstitution with (1) and (2). Thus, the hybridization of (1) and (2) with (4) presumably distorts the segments A and B leading to the dissociation of the surface-confined biocatalytic supramolecular complex.

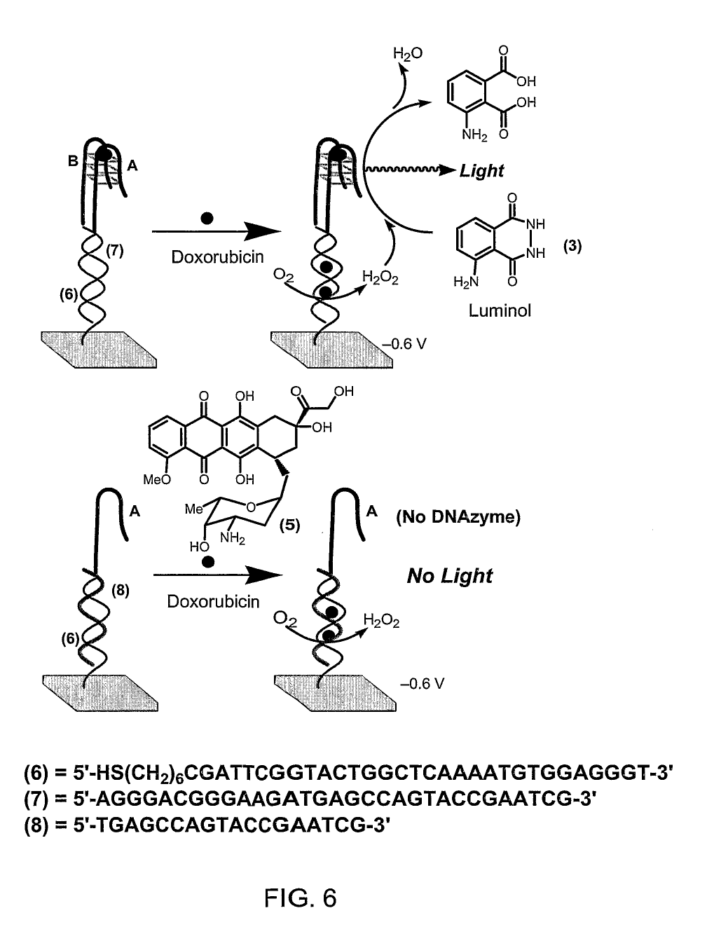

A further surface confined biocatalytic system for the generation of chemiluminescence in the presence of the DNAzyme was designed by the in situ generation of H₂O₂. Previous studies have demonstrated that the intercalation of doxorubicin, (5), into the double-stranded DNA associated with an electrode, allows the electrocatalyzed reduction of O₂ to H₂O₂ by the intercalated quinone, and the subsequence light emission in the presence of HRP and luminol.^{[9]} FIG. 6 shows the assembly of the DNAzyme system on an electrode for the biocatalyzed generation of chemiluminescence, and its application for the analysis of a nucleic acid. The thiolated nucleic acid, (6), that includes the nucleic acid component “A” of the DNAzyme, is assembled on the electrode. The hybridization of the non-enzymatic part of (6) with the complementary part of the nucleic acid (7), that includes the segment “B” of the DNAzyme, yields the interface that binds hemin and generates the peroxidase mimicking DNAzyme. The intercalation of doxorubicin, (5), to the double-stranded DNAzyme produces the bioelectrocatalytic interface for the electrocatalyzed light emission. The electrocatalyzed reduction of (5) produces H₂O₂ and the DNAzyme catalyzes the emission of light in the presence of luminol. Microgravimetric quartz crystal microbalance experiments indicate that the surface coverage of the thiolated nucleic acid (6) is 9.5×10⁻¹² mole·cm⁻², and of the double-stranded nucleic acid structure (6)/(7) is 4.6×10⁻¹² mole·cm⁻². Coulometric assay of doxorubicin (5), response indicated a surface coverage of ca. 2.8×10⁻¹¹ mole·cm⁻². Thus ca. six doxorubicin units are intercalated into each double-stranded DNA.

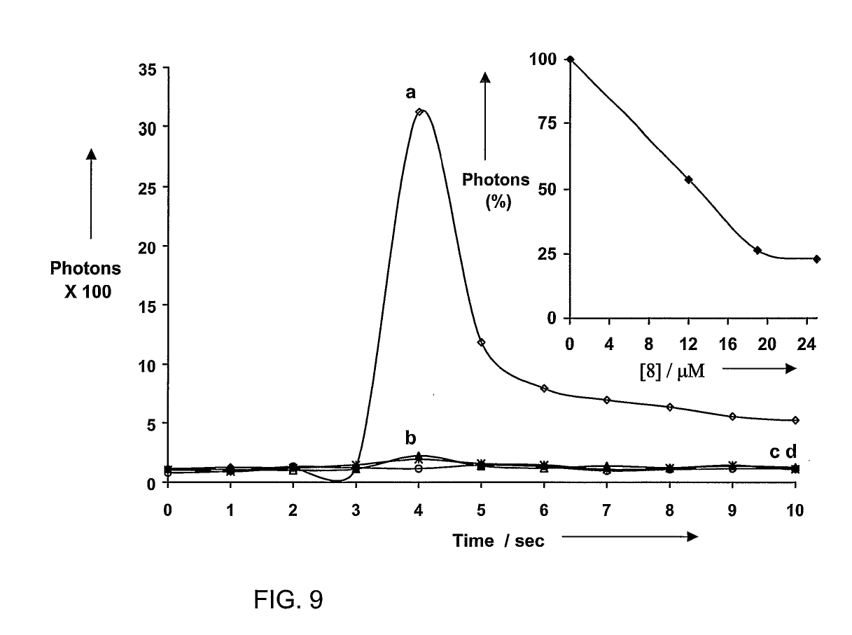

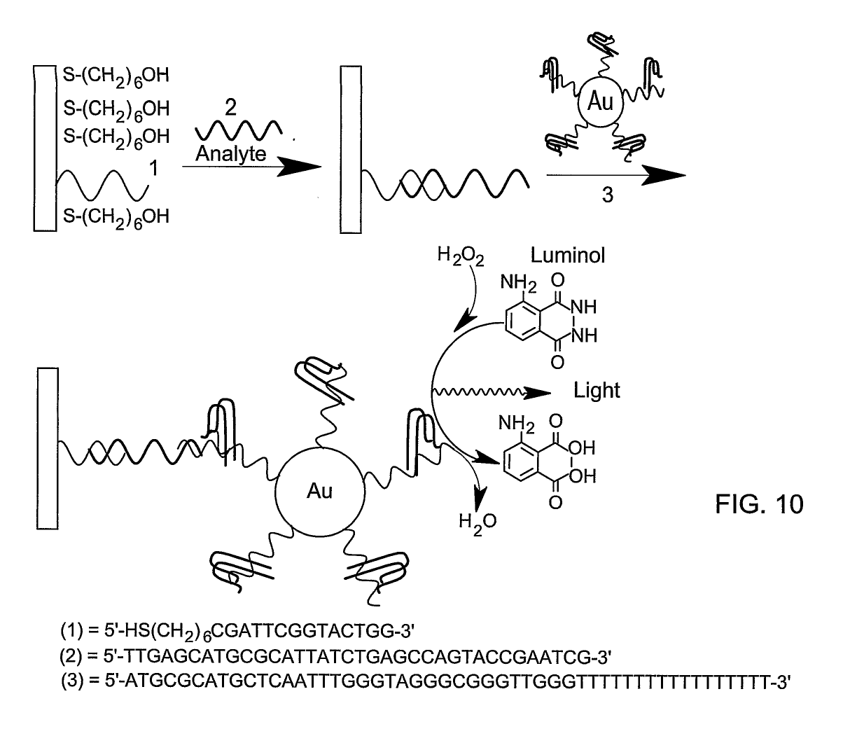

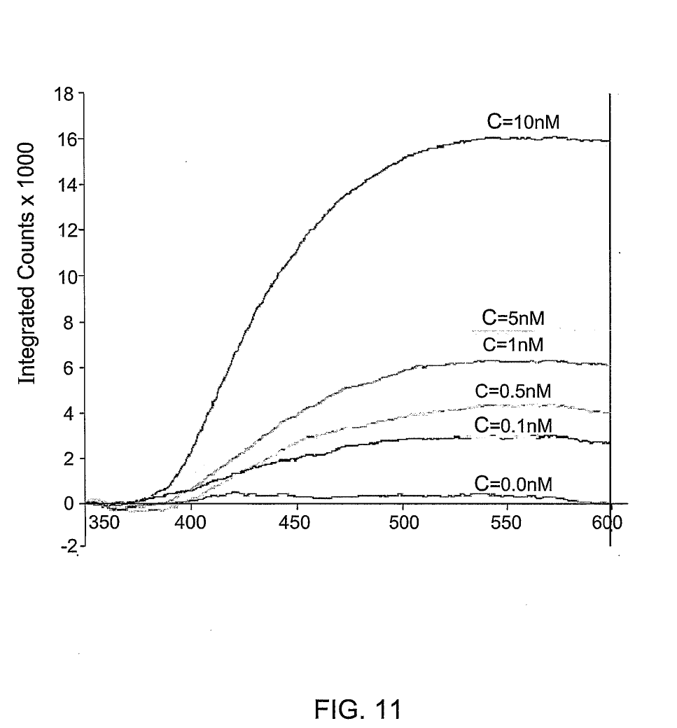

0FIG. 9, curve (a), shows the time-dependent light intensity emitted by the system upon the applying a potential of −0.6 V vs. SCE on the electrode. This potential reduces the doxorubicin associated with the double-stranded DNA on the surface. Doxorubicin mediates the catalyzed generation of H₂O₂ during the reduction process and the electrogenerated H₂O₂ leads to biochemiluminescence in the presence of luminol, (3). Control experiments indicate that no light emission is observed upon application of the same sequence of reactions on the (6)-modified surface without hybridization with (7) (FIG. 9, curve (b)). A further control experiment shows the light emitted from the system consisting of the (6)-functionalized electrode upon interaction with (8) that lacks the segment “B” of the DNAzyme, 2.5 μM, that is further treated with hemin and doxorubicin (5), and subjected to the potential of −0.6 V in the presence of luminol (0FIG. 9, curve (c)). Clearly, the emitted light intensity is negligible, implying that hybridization of (8) with the interface inhibits the formation of the biocatalytic interface for chemiluminescence. Also, the interaction of the mercaptohexanol-functionalized surface with (8) and then with hemin and doxorubicin followed by the application of the potential of −0.6 V vs. SCE in the presence of luminol, (3), did not yield any electrogenerated chemiluminescence (FIG. 9, curve (d)). Thus, the control experiments reveal that the hybridization between (6) and (7) is essential to form the complex with hemin and to intercalate doxorubicin (5), into the double stranded assembly. The electrochemical reduction of the intercalator supplies the H₂O₂ for the DNAzyme and this activates the light emission process. Addition of the nucleic acid (8) that is complementary to the surface-associated nucleic acid (6) competes with (7) towards the hybridization process. Since (8) lacks the “B” part for the self-assembly of the biocatalytic complex with hemin, the light emission in the presence of hybridized (8) should be blocked. 0 FIG. 9, inset, shows the calibration curve that corresponds to the light intensities emitted by the (6)-modified electrode upon hybridization with (7) in the presence of different concentrations of (8), and upon treating the interface with hemin and (5) and applying the reductive potential in the presence of luminol, as described above. The advantages of using DNAzymes as catalytic labels for the analysis of DNA rest, in the enhanced specificity of the analytical protocols. While the use of enzymes and enzyme conjugates always involves non-specific adsorption, the application of nucleic acid catalysts eliminates the phenomenon. One important aspect of the present study is the demonstration that self-assembly of two specific nucleic acids and hemin may yield a supramolecular biocatalytic entity.

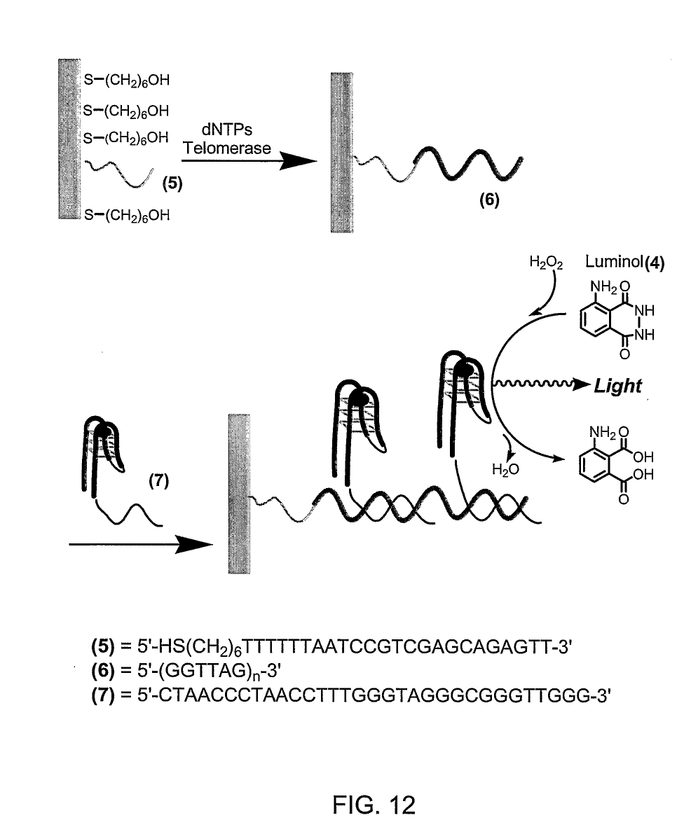

As stated above, previous studies have indicated that a guanine-rich nucleic acid, with the base sequence depicted in FIG. 12, the second part of (7) (beginning with TGGG) is capable of forming a supramolecular G-quadruplex structure with hemin.⁵ The resulting complex exhibited peroxidase-like catalytic activity, and it catalyzed the oxidation of 2,2′-azinobis(3-ethylbenzothiozoline)-6-sulfonic acid, ABTS by H₂O₂. It has now been found that this nucleic acid-hemin complex also reveals peroxidase-like functions towards the oxidation of luminol by H₂O₂ and the generation of chemiluminescence. This property was used to develop a pre-designed DNAzyme label for the amplified detection of DNA.

FIG. 12 depicts the method for the amplified analysis of telomerase activity. The primer (5) is assembled on an Au-surface, and the functionalized surface is interacted with the HeLa cancer cell extract in the presence of a mixture of dNTPs. Since the telomerization leads to a long nucleic acid with constant repeat units (6), the interface may be hybridized with a complementary catalytic nucleic acid complex. The nucleic acid (7) is pre-designed to include the G-rich sequence that forms the catalytic complex with hemin, and a nucleic acid domain that is complementary to the telomere repeat units. The hybridization of the catalytic DNAzyme label with the telomere repeat units associated with the Au-surface, enables the chemiluminescence detection of the telomerase activity by the biocatalytic oxidation of luminol by H₂O₂, and the concomitant light emission. The analysis of telomerase involves two consecutive amplification steps. The first step involves the hybridization of a plurality of catalytic entities to the telomere and the second includes the catalytic DNAzyme that generates numerous photons as a result of a single telomere formation.

The system assembled on the gold surface was characterized by quartz crystal microbalance experiments. The immobilization of (5) on the Au/quartz surface resulted in a frequency change of −40 Hz that translates to a surface coverage of 7.9×10⁻¹² mole·cm⁻² of (5). The telomerization occurring upon the treatment of the functionalized surface with a cell lysate (10,000 cells) in the presence of dNTPs leads to a frequency decrease of 52 Hz, and this translates to a coverage of 6.9×10⁻¹¹ telomere units·cm⁻². That is, an average of ca. 9 telomere units are linked to each primer associated with the surface (this frequency change corresponds to the incorporation of 54 bases into each primer linked to the electrode). The association of the catalytic (7)/hemin label with the surface further decreases the crystal frequency by 50 Hz, indicating a surface coverage of ca. 1.3×10⁻¹¹ mole·cm⁻² or the binding of ca. two DNAzyme units to each telomeric primer.

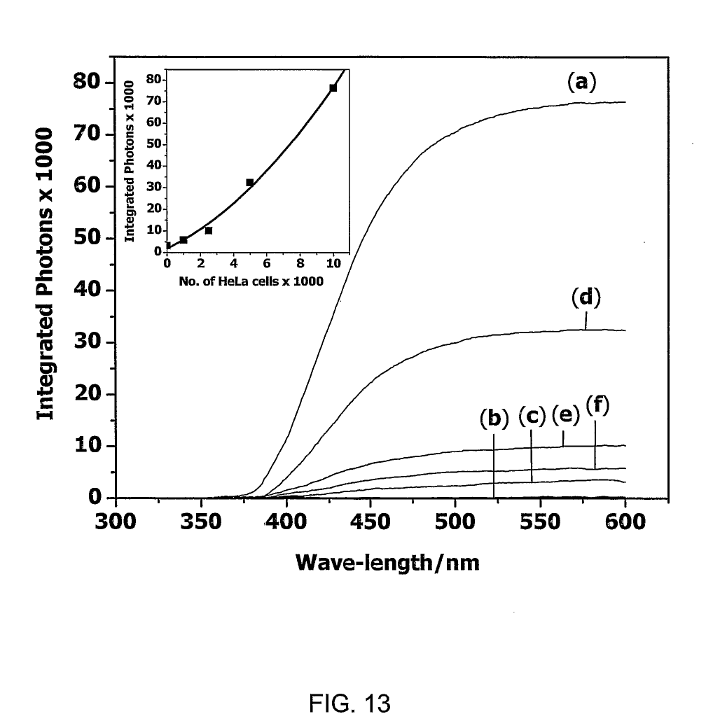

FIG. 13, curve (a), shows the integrated light intensity emitted from the system upon analyzing the telomerase activity originated from 10,000 cells. A control experiment (curve (b)) revealed that upon analysis of heat-treated HeLa cells (85° C. for 10 min) according to the scheme of FIG. 12, no light emission is observed. That is, the thermal deactivation of the telomerase in the HeLa cells prevents telomerization, and the subsequent hybridization of (7) and the biocatalyzed generation of chemiluminescence is inhibited.

Furthermore, this control experiment demonstrates the advantages and utility of the DNAzyme as a label for the amplified detection of DNA. The fact that no chemiluminescence is generated by the heat-treated cells implies that no non-specific interference takes place in the system. Thus, even if cell ingredients bind non-specifically to the surface, their affinity to the catalytic DNAzyme is negligible. Also, the treatment of the telomere units on the surface with hemin leads only to a negligible generation of chemiluminescence, FIG. 13, curve (c). Thus, although the telomere units include G-bases, no biocatalytic complex is generated with hemin, and only the base sequence of (7) is specific to generate with hemin the DNAzyme of peroxidase activity. As the telomerization is controlled by the content of telomerase in the cell lysate samples, the amount of hybridized DNAzyme label, and the intensity of emitted light, should relate to the concentration of cancer cells. FIG. 13 shows the integrated light intensity emitted from the system analyzing variable numbers of HeLa cells (curves (d) to (f)). As expected, the chemiluminescence decreases as the content of HeLa cells in the sample is lower. FIG. 13, inset, shows the calibration curve that corresponds to the emitted light intensity as a function of the number of cells that are analyzed. The detection limit in this experiment corresponds to ca. 1000 HeLa cells in the analyzed sample.

In conclusion, the present study has revealed the novel functions of a hemin-nucleic acid supramolecular complex as a DNAzyme that reveals peroxidase-like chemiluminescence activities. Besides the fundamental interest in the DNAzyme activities of the systems, the systems have important practical implications since the chemiluminescence DNAzyme may act as an internal nucleic acid biocatalytic label for DNA sensing. That is, one may design protein-less amplified DNA detection scheme using chemiluminescence as a transduction means.

1. A method for the determination of an analyte in a sample, said method comprising:
(a) providing a catalytic polynucleotide;
(b) contacting said catalytic polynucleotide with said sample so that the catalytic polynucleotide may bind to the analyte;
(c) providing assay conditions such that said catalytic polynucleotide produces an optically detectable signal in the presence of the analyte; and
(d) detecting said signal, thereby determining the presence of the analyte in the sample.

2. The method of claim 1 wherein said catalytic polynucleotide is a DNAzyme.

3. The method of claim 1 wherein said catalytic polynucleotide is capable of peroxidase activity.

4. The method of claim 3 wherein said catalytic polynucleotide is complexed with hemin.

5. The method of claim 1 wherein said optically detectable signal is produced by a light emitting reaction.

6. The method of claim 5 wherein said light emitting reaction is produced using luminol as a substrate.

7. The method of claim 1 wherein said optically detectable signal is produced by production of a calorimetric product.

8. The method of claim 7 wherein said colorimetric product is produced using the substrate 2,2′-azinobis(3-ethylbenzothiozoline)-6-sulfonic acid (ABTS).

9. The method of claim 1 further comprising the following step:
(e) comparing the optically detectable signal detected in step
(d) with a calibration scale, thereby quantifying the amount of analyte in the sample.

10. The method of claim 1 wherein the analyte is immobilized to a solid surface.

11. The method of claim 10 wherein the analyte is one member of a complex forming group, the method comprising:
(a) immobilizing the analyte on a solid surface;
(b) providing a catalytic polynucleotide bound to another member of said complex forming group;
(c) contacting the catalytic polynucleotide with the solid surface under conditions allowing binding between the two members of the complex forming group;
(d) removing unbound catalytic polynucleotide;
(e) providing assay conditions to allow the catalytic polynucleotide to catalyze a reaction yielding an optically detectable signal; and
(f) detecting said optically detectable signal, thereby detecting the presence of the analyte in the sample.

12. The method of claim 1 wherein a plurality of catalytic polynucleotides are bound to a bead-like particle.

13. The method of claim 1 wherein the analyte is a nucleic acid sequence.

14. The method of claim 13 wherein said analyte is a telomere repeat unit.

15. A method according to claim 1 for the detection of telomerase in a sample, the method comprising:
(a) providing a primer for telomerase activity immobilized on a solid surface;
(b) contacting the sample with said solid surface in the presence of deoxynucleoside triphosphoric acids (dNTP's), under conditions enabling formation of a telomere repeat unit;
(c) adding a catalytic polynucleotide, attached to a sequence complementary to the telomere repeat unit under conditions that allow hybridization of said sequence to the telomere repeat unit;
(d) removing unbound catalytic polynucleotide;
(e) providing substrates for the catalytic polynucleotide to produce an optically detectable signal; and
(f) detecting said signal, the signal indicating the presence of telomerase in the sample.

16. A method for detection of an analyte being one member of a complex forming group in an assay sample, the method comprising:
(a) providing a pre-catalytic polynucleotide comprising a catalytic polynucleotide moiety attached to an inhibitory moiety comprising another member of the complex forming group, said inhibitory moiety in the absence of the analyte sterically hindering the catalytic activity of the catalytic polynucleotide while in the pre-catalytic complex, and said steric hindrance being removed upon binding of the inhibitory moiety to the analyte;
(b) contacting said pre catalytic complex with said assay sample under binding conditions;
(c) providing assay conditions which allow the catalytic polynucleotide to catalyze a reaction yielding an optically detectable signal; and
(d) detecting said signal, thereby detecting the presence of the analyte in the assay sample.

17. A method according to claim 16 for the detection of telomerase in a sample, the method comprising:
(a) providing a pre-catalytic polynucleotide comprising a catalytic polynucleotide attached to an inhibitory moiety, said inhibitory moiety comprising a sequence which is complementary to a telomere repeat unit, the inhibitory moiety, in the absence of the telomere repeat unit, inhibiting the catalytic activity of the catalytic polynucleotide while in the pre-catalytic polynucleotide, the pre-catalytic polynucleotide further comprising a primer for telomerase elongation;
(b) contacting the pre-catalytic polynucleotide with the sample in the presence of dNTPs and under conditions enabling formation of one or more telomere repeat units;
(c) providing substrates for the catalytic polynucleotide; and
(d) detecting an optically detectable signal of the catalytic polynucleotide, detection of the signal being indicative of the presence of telomerase in the sample.

18. A method for detection of telomerase activity in a sample the method comprising:
(a) providing a primer for telomerase activity immobilized on a solid surface;
(b) contacting the sample with the immobilized primer in the presence of dNTP's, under conditions enabling formation of a telomere repeat unit;
(c) adding a catalytic polynucleotide, attached to a sequence complementary to the telomere repeat unit;
(d) removing unbound catalytic polynucleotide;
(e) providing substrates for the catalytic polynucleotide; and
(f) detecting the presence of catalytic products of the catalytic polynucleotide, the products indicating the presence of telomerase activity in the sample.

19. A method for detection of the presence of catalytically active telomerase in a sample, the method comprising:
(a) providing a pre-catalytic polynucleotide comprising a catalytic polynucleotide attached to an inhibitory moiety, said inhibitory moiety comprising a sequence which is complementary to a telomere repeat unit, the inhibitory sequence, in the absence of the telomere repeat unit, inhibiting the catalytic activity of the catalytic polynucleotide while in the pre-catalytic polynucleotide, the pre-catalytic polynucleotide further comprising a primer for telomerase elongation;
(b) contacting the pre-catalytic polynucleotide with the sample in the presence of dNTPs and under conditions enabling primer elongation by telomerase;
(c) providing substrates for the catalytic polynucleotide; and
(d) detecting the presence of catalytic products of the catalytic polynucleotide, detection of the products being indicative for the presence of telomerase in the sample.

20. The method of claim 19 comprising the further step between steps (b) and (c) of providing a co-factor required for the catalytic polynucleotide activity.
